(19) United States
(12) Patent Application Publication
(10) Pub. No.: US 2019/0112574 A1
(43) Pub. Date: Apr. 18, 2019

(54) Culture method for enhancing the activity of stem cells, method of preparing formulation for treating central nervous diseases, and method of treating central nervous diseases

(71) Applicant: University of Macau, Taipa (CN)

(72) Inventors: Ren-He Xu, Taipa (CN); Bin Jiang, Taipa (CN); Li Yan, Taipa (CN); Enqin Li, Taipa (CN)

(21) Appl. No.: 16/047,471

(22) Filed: Jul. 27, 2018

(30) Foreign Application Priority Data
Oct. 17, 2017 (CN) 201710965623.6

(51) Int. Cl. C12N 5/074; C12N 5/0775; A61K 35/28; A61K 9/00; A61P 25/00

(52) U.S. Cl. CPC C12N 5/0607; C12N 5/0663; A61K 35/28; C12N 2523/00; A61P 25/00; C12N 2513/00; A61K 9/0085

(57) ABSTRACT

A culture method for enhancing the activity of stem cells, and a method of preparing a formulation for treating central nervous diseases, and a method of treating central nervous diseases are provided. In some aspects, stem cell clusters are cultured for 3 to 11 days in a sealed container filled with a culture medium, under a condition in which no oxygen and carbon dioxide are supplied. By placing stem cells in a sealed container to stabilize the properties and enhance the functions thereof under a room temperature condition, the stemness and apoptosis resistance of the stem cells are improved, thereby significantly improving the therapeutic effect of the stem cells after transplantation into a human body.

### CROSS-REFERENCE TO RELATED APPLICATION

This application claims priority to Chinese Patent Application No. 201710965623.6, filed on Oct. 17, 2017 with the Chinese Patent Office and entitled “Culture Method for Enhancing the Activity of Stem Cells, Use Thereof, and Formulation for Treating Central Nervous Diseases, Method of Preparing the Same, and Method of Treating Central Nervous Diseases,” the disclosure of which is hereby incorporated herein in its entirety by this reference.

### TECHNICAL FIELD

The application relates to the field of cell biology and, in particular, to a culture method for enhancing the activity of stem cells, use thereof, and a formulation for treating central nervous diseases and a method of preparing the same.

### BACKGROUND

The central nervous system, including the brain and spinal cord, is the most important system for the sensory, conduction and control functions of higher organisms (e.g., humans). Humans' learning, emotion, and thinking are all intensively happening in the brain, and the spinal cord controls the transmission of information into various organs of the entire body, the maintenance of functions of various organs, body movements, and so on. There are a variety of pathogenetic forms and pathogenic causes regarding lesions of the central nervous system. Once a lesion occurs in the central nervous system, it may affect the functions of tissues and organs if it is mild, or it may be life-threatening if it is severe. Moreover, many cases may transform into chronic diseases that are incurable, even by long-term therapy, harm the normal life of the patients, have a high disability rate, seriously threaten human health, and increase the burden on the society.

Cells are the basic unit of life, and the central nervous system is mainly composed of spongy tissues consisting of neuronal cells and neural supporting cells. A variety of degenerative nervous system diseases, such as Parkinson's disease, Alzheimer's disease, idiopathic inherited Huntington's chorea, or the like, are caused due to the aging and disappearance as well as lacking replenishment of normal neuronal cells, thereby resulting in atrophy and lesions of related brain functional nuclei, and ultimately leading to a movement dysfunction or dementia and numbness of the patient. Therefore, transplantation of healthy, active neuronal cells is an effective means for curing such diseases. In addition, mesenchymal stem cells are also functional cells widely used in the treatment of nervous system diseases, which are widely distributed in bone marrow, dental pulp, fat, placenta, and umbilical cord blood. The mesenchymal stem cells are widely used in various immune diseases and spinal cord injury diseases as they have low immunogenicity, inhibit the immune response, regulate the immune function, and have a nourishing effect and an effect of promoting angiogenesis. In the treatment of cerebral palsy, cerebral infarction and demyelinating disease, the mesenchymal stem cells are found to have a remarkable effect in inhibiting the immune response, protecting neurons, and secreting basic fibroblast growth factors and neurotrophic factors to nourish and promote the regeneration and functional recovery of endogenous neurons, and promote vascular regeneration and circulation at lesion sites, which can significantly ameliorate the symptoms and body functions of patients. Therefore, treating the central nervous system diseases with stem cells presents a tangible basis, a high practicability, and meets urgent medical and social needs.

The repeated enzymatic digestion and dissociation prior to being used and the loss due to transportation as experienced by the current stem cells lead to reduced cell quality or even cell apoptosis. During use of stem cells for infusion and transplantation, stem cells that have been dissociated into single cells are transplanted either intravenously or orthotopically for the lesions. However, after the transplantation, a large number of stem cells easily undergo embolization in blood vessels or agglomeration at the lesion sites, leading to apoptosis of a large number of the transplanted stem cells and release of various apoptotic bodies and inflammatory factors, and eventually leading to weakening or even deterioration of the therapeutic effect. Therefore, it is an important technical aspect to seek a suitable method for treating and transplanting stem cells, so as to meet the stem cell therapy.

### BRIEF SUMMARY

This application relates to a culture method for enhancing the activity of stem cells, method(s) of preparing formulation(s) for treating central nervous diseases, and method(s) of treating central nervous diseases.

The applicant has found in study that under a low temperature (room temperature) state, both the growth and metabolism of stem cells are reduced, but their stemness and resistance to apoptosis will be enhanced.

An object of the present disclosure is to provide a culture method for enhancing the activity of stem cells, which can significantly improve the stemness and apoptosis resistance of stem cells.

Another object of the present disclosure is to provide use of the culture method for enhancing the activity of stem cells, which can be used for preparing a formulation for stem cell therapy, and which allows the stem cells to further adapt to a low oxygen environment and have a good resistance to apoptosis after being transplanted.

A further object of the present disclosure is to provide a method of preparing a formulation for treating central nervous diseases, so as to prepare a formulation having better resistance to apoptosis and to treat central nervous diseases.

A further object of the present disclosure is to provide a formulation prepared by the above preparation method.

A further object of the present disclosure is to provide a method of treating central nervous diseases that can significantly ameliorate the symptoms and recover the motor function of a sick body.

Embodiments of the present disclosure are implemented as follows:

A culture method for enhancing the activity of stem cells, wherein stem cell clusters are cultured for 3 to 11 days in a sealed container, filled with a culture medium, and under conditions where no oxygen and carbon dioxide are supplied. Preferably, the culturing is performed for 3 to 7 days.

Use of the culture method for enhancing the activity of stem cells described above, in the preparation of a stem cell formulation.

A method of preparing a formulation for treating central nervous diseases, wherein neural stem cells or mesenchymal stem cells are cultured according to the culture method described above, and the obtained stem cell clusters are resuspended in a saline injection solution after the culture medium is removed therefrom.

A formulation for treating central nervous diseases, prepared by the method of preparing a formulation for treating central nervous diseases described above.

A method of treating central nervous diseases, wherein stem cells are processed according to the culture method described above, and then injected intrathecally or orthotopically into the brain, wherein the stem cell is one or both of mesenchymal stem cell and neural stem cell.

The embodiments of the present disclosure have the following beneficial effects:

The culture method for enhancing the activity of stem cells according to the present disclosure not only significantly improves the stemness and apoptosis resistance of stem cells, but also ensures a higher viability of stem cells. Stem cells processed by this method can be used for preparing a corresponding stem cell formulation and for treating corresponding diseases. For example, in the present disclosure, the culture method described above is used to prepare a formulation for treating central nervous diseases, which may be prepared as a standard product, or may be prepared in situ when in use. Having been processed in a low-oxygen environment, the stem cells have high resistance to apoptosis. After being transplanted into a sick body, the stem cells will not be subjected to apoptosis due to the low-oxygen environment in the sick body, but still maintain good stemness and significantly improve the therapeutic effect.

The method of treating central nervous diseases according to the present disclosure can significantly improve the apoptosis resistance of neural stem cells or mesenchymal stem cells after transplantation into lesions, so that the stem cells can maintain higher stemness and better exert various functions to improve the therapeutic effect.

### BRIEF DESCRIPTION OF THE DRAWINGS

For illustrating technical solutions of embodiments of the disclosure more clearly, drawings required for use in the embodiments will be introduced briefly below. It is to be understood that the drawings below are merely illustrative of some embodiments of the present disclosure and, therefore, should not to be considered as limiting the scope of this disclosure. It would be understood by those of ordinary skill in the art that other relevant drawings could also be obtained from these drawings without any inventive effort.

FIG. 1 is a flowchart of a test example of the disclosure; and

FIG. 2 is a diagram of test results obtained by the test example of the disclosure.

### DETAILED DESCRIPTION

In order to make the, technical solutions of the embodiments of the disclosure more clear, the technical solutions of the embodiments of the disclosure will be described below clearly and completely. Examples are carried out according to conventional conditions or conditions recommended by the manufacturer if no specific conditions are specified in the examples. Reagents or instruments used, whose manufacturers are not specified, are all conventional products that are available commercially.

The repeated enzymatic digestion and dissociation prior to being used and the loss due to transportation as experienced by the current stem cells lead to reduced cell quality or even cell apoptosis. During use of stem cells for infusion and transplantation, stem cells that have been dissociated into single cells are transplanted either intravenously or orthotopically for the lesions. However, a large number of stem cells easily undergo embolization in blood vessels or agglomeration at the lesion sites, leading to apoptosis of a large number of the transplanted stem cells and release of various apoptotic bodies and inflammatory factors, and eventually leading to weakening of the therapeutic effect.

It has been found that this situation is caused because the stem cells are cultured in an environment that is highly nutritious and contains 21% oxygen prior to transplantation, where the stem cells have an intense metabolism and proliferation. However, after the transplantation, the stem cells are brought into an environment that is low in oxygen (less than 13%) or even anoxic, and in which nutrients and sugar cannot be supplied as adequately as in the culture flask, resulting in a large number of stem cells easily undergoing embolization in blood vessels or agglomeration at the lesion sites, leading to apoptosis of a large number of the transplanted stem cells and release of various apoptotic bodies and inflammatory factors, and eventually leading to weakening or even deterioration of the therapeutic effect.

On this basis, provided is a culture method for enhancing the activity of stem cells, wherein stem cell clusters are cultured for 3 to 11 days in a sealed container, filled with a culture medium, under a condition in which no oxygen and carbon dioxide are supplied; and preferably, the culturing is performed for 3 to 7 days. Here, the activity of stem cells refers to stemness and/or apoptosis resistance of the stem cells.

Specifically, the culture method described above is applicable to neural stem cells, mesenchymal stem cells, pluripotent stem cells, adipose stem cells, muscle stem cells, and cardiac stem cells.

In some embodiments, the stem cell clusters are obtained by aggregating the stem cells under a suspension culture condition to form cell clusters. For example, the stem cells may be suspension-cultured in an incubator at 37° C. for 24-48 hours and aggregated to form cell clusters, or the stem cells may be suspension-cultured under a condition of 20° C. to 37° C. and spontaneously aggregate to form cell clusters.

Currently, the production of stem cells depends on a high standard of environment, including cleanliness, humidity, temperature and a proper pH maintained by gases. In general, the operation of stem cells is usually carried out in a culture room of cleanliness Class 10,000 and a sterile operation platform of cleanliness Class 100. Stem cells are grown in an incubator at 37° C., which has a humidity of 90% and a carbon dioxide of 5%, by the way of 2D adherent culturing, and are amplified by digestion and passage. This growth pattern is relatively slow, and requires the use of trypsin to digest and collect cells before each use of the stem cells, which easily destroys cell connections among cells and loses extracellular matrix, resulting in reduction of cell activity. In the case of long-distance transportation of a large number of stem cells, the stem cells need to be kept frozen and, thereafter, still have to undergo tedious procedures such as thawing, recovery and amplification. The repeated enzymatic digestion and dissociation prior to being used and the loss due to transportation as experienced by the current stem cells lead to reduced cell quality or even cell apoptosis.

Therefore, preferably, the stem cells are cultured by using a 3D suspension culture system to form cell clusters. The preparation of a large number of stem cells by using a 3D suspension system facilitates the quality control and the collection, and avoids the multiple digestion processes under the 2D condition.

Cell clusters (uniform spheroids) of different sizes are prepared according to different numbers of stem cells and, preferably, the size of the spheroids is between 30 microns and 250 microns (μm).

As the container for culture, a plastic container such as a container made of polyethylene, polypropylene or melamine is preferably used. Moreover, a sealed container may be filled with a culture medium when stem cells are placed in the sealed container, and as a preferable embodiment, the stem cells may be packaged at a controlled amount of 1 million to 2 million cells per milliliter of culture medium.

After packaging, the sealed container is preferably placed under an environment at 10° C. to 35° C. and protected from exposure to intense light. At low temperature outdoors, such as a snowfield that is not at room temperature, the cell container may be placed in a backpack, and the temperature in the backpack is made to be close to the temperature of a body surface of a human, such that the cell container is maintained in an environment at 10° C. or higher.

The stem cells cultured in the way described above have good stemness and resistance to apoptosis; thus, the method described above can be applied to prepare a stem cell formulation for treating diseases of higher organisms. The stem cells processed in this way will not undergo embolization in blood vessels or agglomeration at the lesion sites after being transplanted into a sick body, so that the functions of nourishment and protection as well as regeneration promotion of the stem cells are served, and the cell therapy of immune diseases, injury diseases, degenerative diseases and the like in the central nervous system is fulfilled, with the effects being remarkable. The disclosure has immeasurable scientific and medical economic benefits.

Provided is a method of preparing a formulation for treating central nervous diseases, wherein neural stem cells or mesenchymal stem cells are cultured according to the culture method as described above, and the obtained stem cell clusters are resuspended in a saline injection solution after the culture medium is removed therefrom. A finished stem cell formulation can be prepared according to this method, and stored by suitable storage and transportation methods for treating central nervous diseases of higher organisms.

It should be noted that, in addition to being prepared as a finished product, the formulation may be prepared temporarily during therapy. For example, after being processed in the way described above, the stem cells are transported to a destination by a proper storage method, and then resuspended in a saline injector solution before being used for a sick body.

With the formulation for treating central nervous diseases prepared by the above method, stem cells have high resistance to apoptosis since they have been processed in a low-oxygen environment. After being transplanted into a sick body, the stem cells will not be subjected to apoptosis due to the low-oxygen environment in the sick body, but still maintain good stemness and significantly improve the therapeutic effect.

Also, provided is a method of treating central nervous diseases, wherein stem cells are processed according to the culture method described above, and then injected into a sick body through intrathecal injection or orthotopical brain injection, wherein the stem cells are either one or both mesenchymal stem cells and/or neural stem cells.

As described previously, having been processed in a low-oxygen environment, the stem cells have stronger stemness and resistance to apoptosis, and will not undergo embolization in blood vessels or agglomeration at the lesion sites after being transplanted into a sick body, thereby exerting the functions of nourishment and protection as well as regeneration promotion of the mesenchymal stem cells or neural stem cells, and achieving the purpose of treating central nervous system diseases.

Embodiments provide a method of treating central nervous system diseases with stem cells, which is a feasible method for transplanting neural stem cells and mesenchymal stem cells, after being processed, into the central nervous system so as to treat a central nervous system disease, comprising three steps: 1) making the neural stem cells and the mesenchymal stem cells form cell clusters by using a suspension culture system; 2) processing the cell clusters formed from the neural stem cells and the mesenchymal stem cells at a room temperature; and 3) directly transplanting the cell clusters formed by the neural stem cells and the mesenchymal stem cells into the central nervous system by a direct intrathecal injection, fulfilling their functions.

Preferably, the stem cells are first spontaneously aggregated to form spheroids by the fact that the stem cells have the property of forming spheroids.

Preferably, in the method for clustering the stem cells, cell clusters of different sizes are prepared according to different numbers of mesenchymal stem cells, and preferably the size of the spheroids is between 30 microns and 250 microns.

Preferably, the cell clusters are in the form of uniform spheroids.

Preferably, the clustering of the stem cells is required to be carried out by culturing the stem cells in an incubator at 37° C. for 24 to 48 hours; or the stem cell clusters can also be formed spontaneously under a condition of 20° C. to 37° C.

Preferably, the incubator has a carbon dioxide content of 5% and a humidity of greater than 80%.

Preferably, the proliferative and metabolic rate of the mesenchymal stem cells, in clusters of high cell density, are naturally reduced by taking the advantage of the principle of cell contact inhibition, thereby achieving the purpose of reducing oxygen consumption and nutrient consumption.

Preferably, the stem cell spheroids are placed under a room temperature condition for 3 to 7 days to enhance their activity according to a phenomenon that the stem cells have increased stemness and increased activity under low-temperature condition.

Preferably, the low-temperature condition is a condition of a room temperature.

Preferably, the stem cell clusters are packaged and stored in a sealed container, and preferably, the container is a plastic container made of raw materials such as polyethylene, polypropylene and melamine, and the stem cells are packaged at a density of 1 million to 2 million cells per milliliter of medium; and energy consumption is further reduced to adapt to a low-oxygen environment.

Preferably, during transportation, it is only required to place a cell container at a room temperature ranging from 10° C. to 3520 C. and protect the cell container from exposure to intense light, a high temperature or a low temperature. At a low temperature outdoors, such as a snowfield that is not a room temperature, the cell container may be placed in a backpack, and a temperature in the backpack is made to be close to the temperature of a body surface of humans, such that the cell container is maintained in an environment at 10° C. or higher.

Preferably, the high temperature is greater than 42° C., and the low temperature is less than 020 C.

Preferably, the stem cell spheroids can be directly applied for intrathecal injection or brain transplantation after a centrifugation and a removing of the culture medium.

Preferably, the method not only can be applied on the mesenchymal stem cells and the neural stem cells, but also applied directly on pluripotent stem cells and other types of stem cells such as adipose stem cells, muscle stem cells, cardiac stem cells, and so on.

Preferably, the mesenchymal stem cells are derived from bone marrow and a differentiating of pluripotent stem cells.

The features and functions of the present disclosure will be described below in further detail with reference to examples.

### EXAMPLE 1

Two strains of mesenchymal stem cells (one being differentiated from human pluripotent stem cells CT3, and the other being isolated from a donated bone marrow blood) were selected.

The two strains of cells were cultured in a normal cell culture environment (37° C., 5% carbon dioxide, 90% humidity) to reach a confluence of 80%. Then, experiments were carried out according to the following steps:

1. the cells were taken out, from which a culture medium was removed, and the cells were washed with 1 ×PBS twice;

2. the cells were digested with 0.05% trypsin for 3 minutes;

3. the cells were collected with a stem cell culture medium (low glucose DMEM, 20% serum, 1% nonessential amino acid and 5% L-glutamine);

4. the cell concentration was adjusted to 8×10⁵ cells/ml, and stem cell spheroids were prepared using hanging drop method, at 25 microliters per drop;

5. the culture plate was placed in a normal cell culture environment for culturing for 48 hours;

6. small spheroids of stem cells were collected from hanging drops;

7. centrifuging at 1000 rpm for 3 minutes, and the supernatant was removed;

8. a freshly stored culture medium was added to resuspend the cells, such that the density of cells was adjusted to 2 million cells per milliliter of the culture medium;

9. the cells were added into a 1.5 ml plastic centrifuge tube, which was sealed by a paraffin film and kept from exposure to the light; this day was marked as day 0, and the cells were stored at room temperature (20° C.) for 7 days;

10. on day 7, the cells were centrifuged; the supernatant was removed; and the cells were washed with 1×PBS twice;

11. the cells were digested with 0.05% trypsin for 3 minutes;

12. the cells were resuspended with a stem cell culture medium (low sugar DMEM, 20% serum, 1% nonessential amino acid and 5% L-glutamine); and

13. the cells were detected.

The detection results show that both strains of the mesenchymal stem cells resulting from this method have a viability of more than 90% and have a normal cell morphology.

### EXAMPLE 2

The present example was different from Example 1 only in that the density of cells was adjusted to 1 million cells per milliliter of the culture medium in step 8, and the cells were stored for 3 days in step 9.

It is detected that both the mesenchymal stem cells resulting from this method have a viability of more than 90% and have a normal cell morphology.

### EXAMPLE 3

The present example was different from Example 1 only in that the cells were stored for 11 days in step 9.

It is detected that both the mesenchymal stem cells resulting from this method have a viability of more than 90% and have a normal cell morphology.

### EXAMPLE 4

The present example was different from Example 1 only in the culturing mode in step 9. In the present example, the culture plate was placed under a condition at 20° C. for suspension culturing, until the cells spontaneously aggregated to form cell clusters.

### EXAMPLE 5

The present example provides a formulation for treating central nervous diseases, which is prepared by resuspending, in normal saline, the mesenchymal stem cell clusters obtained by the treatments in Example 1 to form an injection solution. The formulation is stored under a condition at 10° C. to 37° C. and protected from light.

### TEST EXAMPLE

14. The mesenchymal stem cell spheroids prepared in Example 1 described above were placed in a backpack and transported at room temperature for 3 days to a monkey farm where the diseased monkeys were located, wherein the diseased monkeys suffered from multiple sclerosis in the spinal cord;

15. the cell spheroids were centrifuged to remove the culture medium and washed with 1×PBS twice;

16. the cells were resuspended in 1 ml of 1×PBS, and sucked into a 1 ml syringe for injection;

17. after the diseased monkeys were anesthetized, 2 ml of cerebrospinal fluid was firstly released by an intrathecal injection needle to prevent an increase in intracranial pressure during injection of the cells;

18. eight diseased cynomolgus monkeys were selected, and the cells provided in step 16 were slowly injected intrathecally into the diseased monkeys, wherein the responses of the diseased monkeys should be observed, and no cynomolgus monkey showed adverse symptoms during the injection in the present test example (it should be noted that the injection should be ceased immediately if any discomfort was observed in the diseased monkeys during the injection);

19. after the injection was completed, the diseased monkeys were placed back into the cages for continuous observation;

wherein it could be observed on the next day after the injection that the diseased monkeys showed an increase in food intake, and coordinated movements and increased activities could be visibly observed on the second day after the injection,

20. various physical data for the diseased monkeys were observed and recorded. The diseased monkeys were sacrificed after 3 months, and central nervous system samples were collected for pathological detection. The detection results were as shown in FIG. 2.

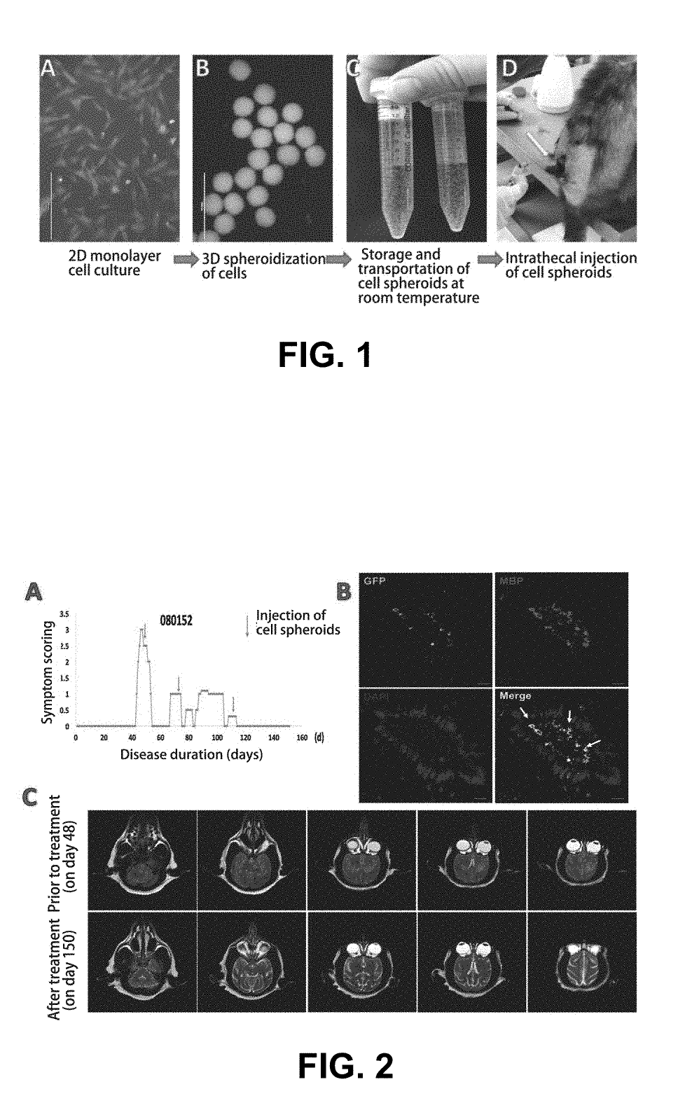

FIG. 1 shows the procedures of the process of this test, referring to FIG. 1: wherein FIG. 1, Panel A, represents mesenchymal stem cells Envy-EMSCs cultured in monolayer, containing green fluorescent protein (GFP);

FIG. 1, Panel B, represents cell spheroids with a diameter of 250 microns prepared from the Envy-EMSCs in step 8;

FIG. 1, Panel C, represents that the Envy-EMSC cell spheroids are packaged in a centrifuge tube, placed under a room temperature condition for 3 days, and transported to a monkey farm in step 14; and

FIG. 1, Panel D, represents a process in which the Envy-EMSC cell spheroids (FIG. 1, Panel C) are directly injected intrathecally into the spinal lumen of cynomolgus monkeys after release of 2 ml of cerebrospinal fluid in step 18.

According to the analysis of FIG. 2, it can be seen from FIG. 2, Panel A, that the symptoms of the diseased monkeys were continuously at low level after stem cells are injected into the monkeys. Here, the scoring criterion is based on the criteria for scoring clinical symptoms of multiple sclerosis in spinal cord (“2010 McDonald diagnostic criteria for MS”).

As can be seen in FIG. 2, Panel B, the result of detection of the diseased monkeys by magnetic resonance imaging (MM) showed that the diseased monkeys were positive for MBP after cell therapy, indicating that Envy-EMSCs were migrated directly to the lesion sites of the brain and transformed into oligodendrocytes.

The diseased monkeys were detected by magnetic resonance imaging (MRI). FIG. 2, Panel C, shows images of brain structures of the diseased monkeys before and after cell therapy, from which it could be seen that there were many lesions (lumps in the figure) in the brain structure before therapy, and cynomolgus monkeys with multiple sclerosis in the spinal cord showed remarkable reduction of brain lesions after the cell therapy, indicating that the inflammation was alleviated, and tissues were repaired and ameliorated.

The embodiments described above are some, but not all, of the embodiments of the disclosure. The detailed description of the embodiments of the disclosure is not intended to limit the scope of the disclosure as claimed, but is merely representative of selected embodiments of the disclosure. All the other embodiments obtained by those of ordinary skill in the art in light of the embodiments of the disclosure without inventive efforts would fall within the scope of the disclosure as claimed.

What is claimed is:

1. A method for enhancing an activity of stem cells, the method comprising:
culturing stem cell clusters for three (3) to eleven (11) days in a sealed container filled with a culture medium, under a condition in which no oxygen and carbon dioxide are supplied.

2. The method according to claim 1, wherein culturing is performed for three (3) to seven (7) days.

3. The method according to claim 1, wherein the stem cells comprise mesenchymal stem cells, neural stem cells, pluripotent stem cells, adipose stem cells, muscle stem cells, and/or cardiac stem cells.

4. The method according to claim 1,
wherein the stem cell clusters are obtained by aggregating the stem cells under a suspension culture condition to form cell clusters, and
wherein the stem cells are cultured with a 3D suspension culture system to form cell clusters.

5. The method according to claim 4, wherein a diameter of the cell clusters is between 30 μm and 250 μm.

6. The method according to claim 4,
wherein the stem cells are suspension-cultured in an incubator at 37° C. for 24-48 hours and aggregated to form cell clusters, or
wherein the stem cells are suspension-cultured under a condition of 20° C. to 37° C. and spontaneously aggregate to form cell clusters.

7. The method according to claim 1, wherein the container is a plastic container, and the plastic container is made of polyethylene, polypropylene or melamine.

8. The method according to claim 1, wherein the stem cells are packaged at an amount of 1 million to 2 million cells per milliliter of culture medium.

9. The method according to claim 1, further comprising:
preparing a stem cell formulation therefrom.

10. A method of preparing a formulation for treating a central nervous disease or diseases, the method comprising:
culturing neural stem cells or mesenchymal stem cells for three (3) to eleven (11) days in a sealed container filled with a culture medium, under a condition in which no oxygen and carbon dioxide are supplied, removing the culture medium therefrom, and then resuspending the obtained stem cell clusters in a saline injection solution.

11. The method according to claim 10, wherein culturing is performed for three (3) to seven (7) days.

12. The method according to claim 10, wherein the stem cells comprise mesenchymal stem cells, neural stem cells, pluripotent stem cells, adipose stem cells, muscle stem cells, or cardiac stem cells.

13. The method according to claim 10,
wherein the stem cell clusters are obtained by aggregating the stem cells under a suspension culture condition to form cell clusters, and
wherein the stem cells are cultured by using a 3D suspension culture system to form cell clusters.

14. The method according to claim 13, wherein a diameter of the cell clusters is between 30 μm and 250 μm.

15. The method according to claim 13,
wherein the stem cells are suspension-cultured in an incubator at 37° C. for 24-48 hours and aggregated to form cell clusters, or
wherein the stem cells are suspension-cultured under a condition of 20° C. to 37° C. and spontaneously aggregate to form cell clusters.

16. A method of treating a subject for central nervous disease(s), the method comprising:
culturing stem cell clusters for three (3) to eleven (11) days in a sealed container filled with a culture medium, under a condition in which no oxygen and carbon dioxide are supplied, and
then injecting the stem cell clusters into the subject through intrathecal injection or orthotopical brain injection,
wherein the stem cells are either one or both of mesenchymal stem cells and neural stem cells.

17. The method according to claim 16, wherein the culturing is performed for three (3) to seven (7) days.

18. The method according to claim 16, wherein the stem cells comprise mesenchymal stem cells, neural stem cells, pluripotent stem cells, adipose stem cells, muscle stem cells, or cardiac stem cells.

19. The method according to claim 16,
wherein the stem cell clusters are obtained by aggregating the stem cells under a suspension culture condition to form cell clusters, and
wherein the stem cells are cultured by using a 3D suspension culture system to form cell clusters.

20. The method according to claim 16, wherein a diameter of the cell clusters is between 30 μm and 250 μm.
